(19) United States
(12) Patent Application Publication
(10) Pub. No.: US 2008/0265512 A1
(43) Pub. Date: Oct. 30, 2008

(54) Hand assisted laparoscopic seal apparatus with a fast recovery foam core

(76) Inventors: Andrew T. Beckman, Cincinnati, OH (US); David E. Havens, Dayton, OH (US); Kristin Muckley, Monroe, OH (US); Matthew C. Everhart, Fairborn, OH (US)

Correspondence Address: WELSH & FLAXMAN LLC, 2000 DUKE STREET, SUITE 100, ALEXANDRIA, VA, 22314, US

(21) Appl. No.: 11/790,885

(22) Filed: Apr. 27, 2007

(51) Int. Cl. F16J 15/00; A61B 1/12

(52) U.S. Cl. 277/301; 604/184

(57) ABSTRACT

A hand assisted laparoscopic seal apparatus. The seal apparatus includes a seal body and a housing supporting the seal body. The seal body includes a core composed of a fast recovery foam and a flexible membrane covering the core.

### BACKGROUND OF THE INVENTION

1. Field of the Invention

The invention relates to laparoscopic devices. In particular, the invention relates to a laparoscopic seal apparatus with a fast recovery foam core.

2. Description of the Prior Art

During laparoscopic procedures, it is often desirable for the surgeon to place his or her hand within the patient in a manner manipulating the instruments positioned within the patient. When this occurs, it is desirable to separate the external environment from the internal portion of the patient. For example, when hand assisted laparoscopic procedures are performed within the abdominal cavity, it is desirable to perform hand exchanges with minimal loss of abdominal pressure.

As such, a need exists for skin mountable seals permitting hand assisted laparoscopic procedures without fear the abdominal pressure will be compromised. The present invention provides such an apparatus.

### SUMMARY OF THE INVENTION

It is, therefore, an object of the present invention to provide a hand assisted laparoscopic seal apparatus. The seal apparatus includes a seal body and a housing supporting the seal body. The seal body includes a core composed of a fast recovery foam and a flexible membrane covering the core.

It is also an object of the present invention to provide a seal apparatus wherein the fast recover foam is a fast recovery open cell foam.

It is another object of the present invention to provide a seal apparatus wherein the flexible membrane is an elastomer embedded flexible spandex matrix.

It is a further object of the present invention to provide a seal apparatus wherein the core is composed of multiple layers of open cell foam.

It is also another object of the present invention to provide a seal apparatus wherein the core is constructed in a disk shape with a central slit shaped and dimensioned to permit the passage of a physician's hand therethrough while also providing a complete seal when the hand is removed.

It is also a further object of the present invention to provide a seal apparatus wherein the flexible membrane is composed of a spandex matrix which has been impregnated with an elastomer resin to reinforce the material and provide a smooth surface for the insertion of a physician's hand through the central slit.

It is still another object of the present invention to provide a seal apparatus wherein the seal is supported in the center of the housing.

It is yet another object of the present invention to provide a seal apparatus wherein the housing includes an annular base member having an inwardly directed inner rim upon which is placed a lower surface of the seal body.

It is a further object of the present invention to provide a seal apparatus wherein the housing also includes an upper closure gasket secured to the seal body and lower gasket member in a manner securing all components of the present seal apparatus together.

It is yet a further object of the present invention to provide a seal apparatus including a retractor coupled to the housing.

It is also an object of the present invention to provide a seal apparatus wherein the flexible membrane is a silicone embedded flexible spandex matrix.

It is another object of the present invention to provide a seal apparatus wherein the core has a thickness of approximately 0.5 inches to approximately 1.5 inches.

It is a further object of the present invention to provide a seal apparatus wherein the core a recovery rate less than approximately 1 second.

It is also an object of the present invention to provide a seal apparatus wherein the core has a density less than approximately 0.5 g/cm².

It is also an object of the present invention to provide a seal apparatus wherein the core has a thickness of approximately 0.5 inches to approximately 1.5 inches, a recovery rate less than approximately 1 second, and a density less than approximately 0.5 g/cm².

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a perspective view of the present seal apparatus.

FIG. 2 is an exploded view of the present seal apparatus.

FIG. 3 is a cross-sectional view of the present seal apparatus.

FIG. 4 is a cross-sectional view of the present seal apparatus showing a hand inserted therethrough.

FIG. 5 is a cross-sectional view in accordance with an alternate embodiment.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as the basis for teaching one skilled in the art how to make and/or use the invention.

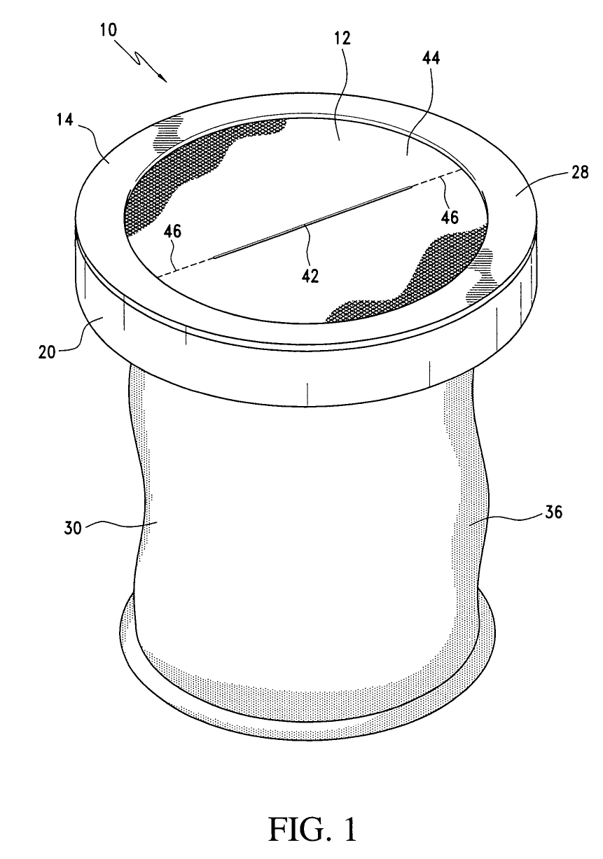

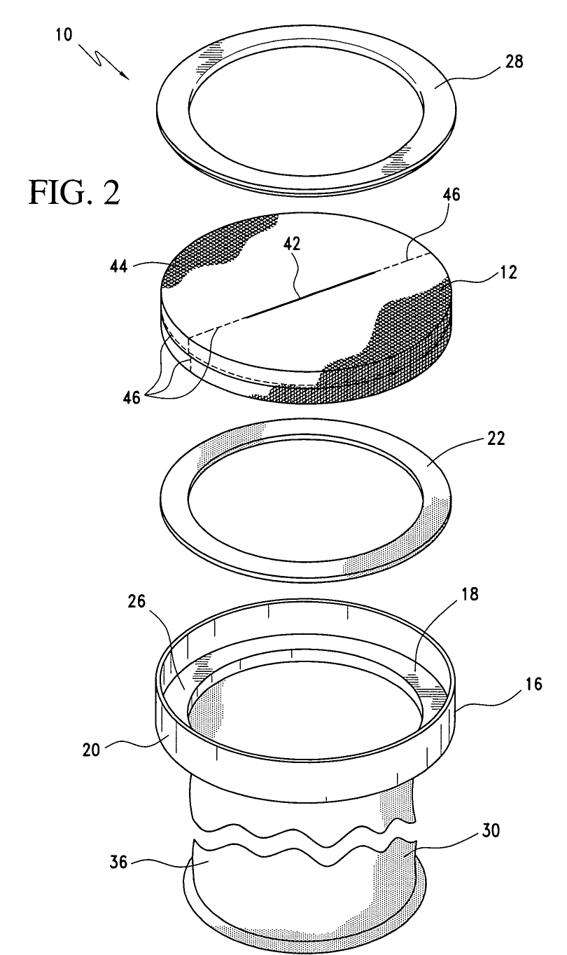

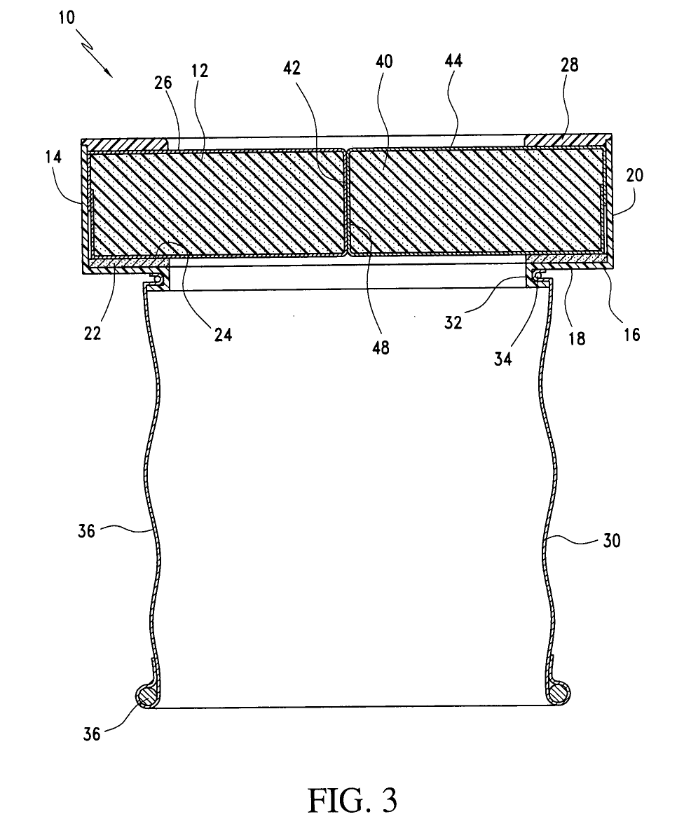

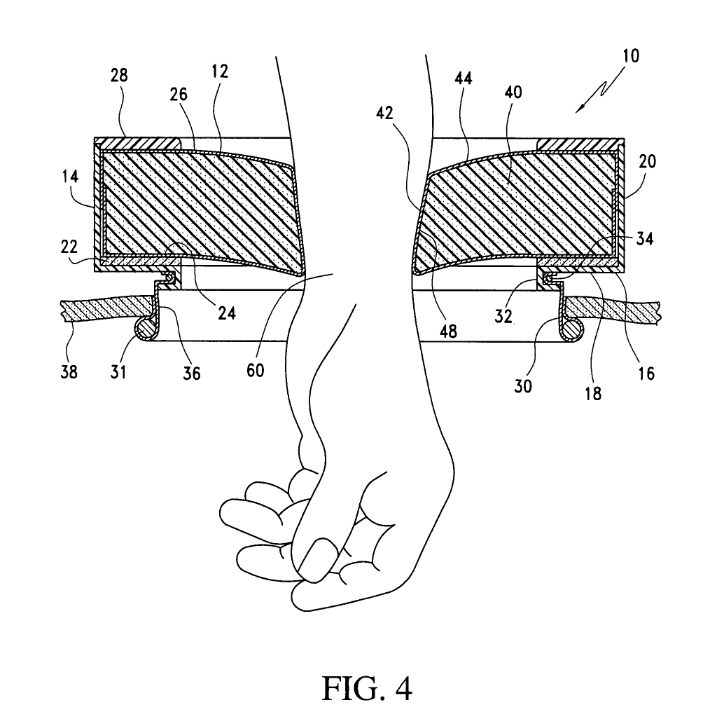

Referring to FIGS. 1 to 4, a seal apparatus 10 for permitting hand assisted laparoscopic procedures is disclosed. The seal apparatus 10 generally employs a single seal body 12 designed to ensure abdominal pressure is not compromised during hand exchanges while hand assisted laparoscopic procedures are performed. Although a single seal body design is disclosed in accordance with a preferred embodiment of the present invention, those skilled in the art will appreciate the concepts underlying the present invention may be applied to apparatuses employing additional seal layers without departing from the spirit of the present invention.

The seal body 12 is shaped and dimensioned to create a gas tight barrier around the surgeon's wrist or forearm 60 when inserted through the seal apparatus 10. It is further shaped and dimensioned to create a gas tight barrier between the interior abdominal space and the external environment when a hand is not inserted through the seal apparatus 10.

Referring to the various figures, the seal body 12 is positioned within a housing 14. In accordance with a preferred embodiment, the housing 14 is made of polyurethane, polycarbonate, and soft elastomers, such as, EPDM (Ethylene Propylene Dimonomer) and SANTOPRENE (that is, a thermoplastic elastomer). While preferred materials are disclosed in accordance with a preferred embodiment, those skilled in the art will appreciate a variety of materials may be used without departing from the spirit of the present invention.

The housing 14 preferably supports the seal body 12 in the center of the housing 14, although noncentric constructions may be employed without departing from the spirit of the present invention. More particularly, the housing 14 includes an annular base member 16. The annular base member 16 includes an inwardly directed inner rim 18 and a circumferentially extending outer border 20. Positioned along the inner rim 18 is a lower gasket member 22 upon which is placed the lower surface 24 of the seal body 12. Positioned directly upon the upper surface 26 of the seal body 12 is an upper closure gasket 28. The upper closure gasket 28 is secured to the seal body 12 and lower gasket member 22 in a manner securing all components of the present seal apparatus 10 together. In accordance with a preferred embodiment, the various components are held together using mechanical attachment structures, such as, press fit rivets, adhesive, welds, or other suitable securing mechanism known to those skilled in the art.

The housing 14 is secured to the skin of an individual by first creating an incision and positioning the housing 14 adjacent the incision. Although a fixedly secured retractor is disclosed in accordance with a preferred embodiment, those skilled in the art will appreciate that where a detachable retractor is used, the retractor should be install first, then the seal cap is attached. Thereafter, a retractor 30 (or wound protector), which is coupled to the housing 14 at housing lower end 32 by an O-ring 34, is inserted into the body cavity. The skirt 36 of the retractor 30 is then drawn about the lower end 32 of the housing 14 with the abdominal wall 38 therebetween. The housing 14 is securely supported on the abdominal wall 38 with the abdominal wall 38 securely held between the housing 14 and the lower retractor ring 31 of the retractor 30.

Although a fixed length retractor is disclosed in accordance with a preferred embodiment of the present invention, it is contemplated adjustable retractors may be used without departing from the spirit of the present invention. For example, an adjustable retractor may be employed which allows for one to “roll-up” the free end of the retractor, or otherwise adjust the length of the retractor, for proper positioning of the present seal apparatus relative to an incision. In addition, and as briefly discussed above, it is contemplated the retractor may be selectively secured to the seal apparatus through the utilization of an attachment ring and attachment technique as disclosed in U.S. patent application Ser. No. 11/607,118, entitled “Hand Assisted Laparoscopic Device”, which is incorporated herein by reference.

More particularly, and in accordance with one technique for the performance of hand assisted laparoscopic procedures, the surgical site is prepared in accordance with conventional standard hospital procedures, making sure the skin is clean and dry. Thereafter, a template is placed over the incision site and an incision line is marked upon the template using a sterile skin marker. As those skilled in the art will appreciate, the glove size of the surgeon dictates the size of the incision. For example, if the surgeon's glove size is 7, a 6.5 to 7 cm incision is usually appropriate. An incision is then made along the marked incision line. The incision size is verified by inserting the surgeon's hand into the abdomen prior to attaching the present seal apparatus. If the incision is too small, the incision is extended on each end as required to maintain the central position of the incision relative to the placement of the present seal apparatus. Thereafter, the retractor is inserted through the incision. Using one's fingers, the lower retractor ring of the retractor is seated evenly under the peritoneum and the area is swept to ensure that tissue is not trapped between the retractor and the abdominal cavity. Thereafter, the housing is pulled down into contact with the skin with the patient's tissue between the retractor and the housing.

With regard to the seal body 12, and in accordance with a preferred embodiment, it is generally composed of a fast recovery, open cell foam (that is, a fast recovery, cellular structure). In particular, for the purpose of the present disclosure, a foam is considered to be a material in which there is interconnection between the cells. As will be discussed below in greater detail, the fast recovery, open cell foam is covered with an elastomer, for example, silicone embedded (or reinforced) flexible spandex matrix.

Although an open cell foam is disclosed in accordance with a preferred embodiment, it is contemplated the seal body may be composed of a honeycomb matrix or other similar structure without departing form the spirit of the present invention. In addition, although the term “foam” is used in describing a preferred embodiment of the present invention, this term is considered to encompass sponges, filters and other resilient cellular structures known to those skilled in the art.

More particularly, the seal body 12 includes a core 40 respectively composed of fast recovery open cell foam. In accordance with a preferred embodiment, the foam is Neoprene or EPDM, although other materials maybe used in accordance with a preferred embodiment. In general, the foam is a porous, low compression set, fast recovery open foam, with a recovery rate less than 1 second and a density less than 0.5 g/cm̂2. In accordance with a preferred embodiment, the foam thickness should be greater than approximately 0.5 inches and preferably approximately 1.5 inches.

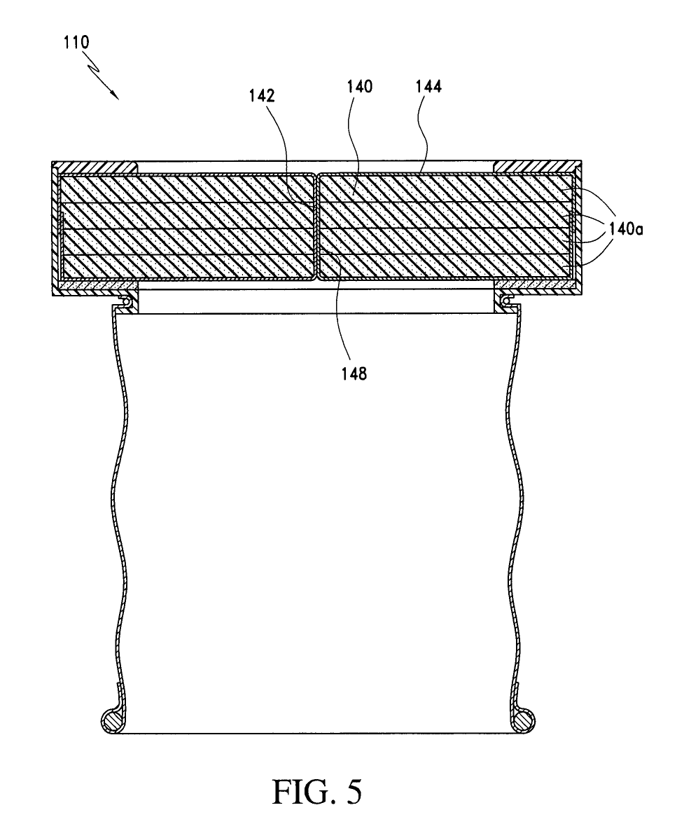

Although a single piece core 40 is disclosed with reference to FIGS. 1 to 4, it is contemplated the core 140 of the seal apparatus 110 may be composed of layers 140a of fast recovery open cell foam secured together via adhesive as shown in FIGS. 5. In accordance with a preferred embodiment, although a preferred foam is disclosed in accordance with a preferred embodiment, other foam materials may be employed without departing from the spirit of the present invention.

The core 40, 140 is generally constructed in a disk shape with a central slit 42, 142 shaped and dimensioned to permit the passage of a physician's hand therethrough while also providing a complete seal when the hand is removed. The core 40, 140 is wrapped in a flexible membrane 44, 144. The flexible membrane 44, 144 is composed of a spandex matrix, that is, a synthetic fiber or fabric made from a polymer containing polyurethane to provide strength, which has been impregnated with silicone resin to prevent pneumo (abdominal pressure) loss and provide a smooth surface for the insertion of a physician's hand through the slit.

In accordance with a preferred embodiment, the flexible membrane 44, 144 is secured about the core 40, 140 by stitching various edges of the membrane 44, 144 together until it completely covers the entire core 40, 140 including the internal wall 48, 148 defining the central slit 42, 142. The stitches 46 (only shown with regard to the embodiment of FIGS. 1 to 4) are then covered in silicone resin to improve the smoothness of the skin.

While an open cell foam is disclosed in accordance with a preferred embodiment of the present invention, the core might be replaced with a gel material or other similar material without departing from the spirit of the present invention.

Construction of the seal body in this manner provides a seal which creates a gas tight barrier around the surgeon's wrist when inserted through the device. The seal body further creates a gas tight barrier between the interior abdominal space and the outside when a hand is not inserted through the device. As a result, the present seal facilitates hand exchanges with minimal loss of abdominal pressure (pneumo).

Although a particular slit construction is disclosed in accordance with a preferred embodiment of the present invention, the slit could be angled, twisted or multidirectional.

While the preferred embodiments have been shown and described, it will be understood that there is no intent to limit the invention by such disclosure, but rather, is intended to cover all modifications and alternate constructions falling within the spirit and scope of the invention.

1. A hand assisted laparoscopic seal apparatus, comprising:
a seal body;
a housing supporting the seal body;
wherein the seal body includes a core composed of a fast recovery foam and a flexible membrane covering the core.

2. The seal apparatus according to claim 1, wherein the fast recover foam is a fast recovery open cell foam.

3. The seal apparatus according to claim 2, wherein the flexible membrane is a elastomer embedded flexible spandex matrix.

4. The seal apparatus according to claim 3, wherein the core is composed of multiple layers of open cell foam.

5. The seal apparatus according to claim 2, wherein the core is constructed in a disk shape with a central slit shaped and dimensioned to permit the passage of a physician's hand therethrough while also providing a complete seal when the hand is removed.

6. The seal apparatus according to claim 5, wherein the flexible membrane is composed of a spandex matrix which has been impregnated with an elastomer resin to reinforce the material and provide a smooth surface for the insertion of a physician's hand through the central slit.

7. The seal apparatus according to claim 1, wherein the seal is supported in the center of the housing.

8. The seal apparatus according to claim 1, wherein the housing includes an annular base member having an inwardly directed inner rim upon which is placed a lower surface of the seal body.

9. The seal apparatus according to claim 8, wherein the housing also includes an upper closure gasket secured to the seal body and lower gasket member in a manner securing all components of the present seal apparatus together.

10. The seal apparatus according to claim 1, further including a retractor coupled to the housing.

11. The seal apparatus according to claim 1, wherein the flexible membrane is a silicone embedded flexible spandex matrix.

12. The seal apparatus according to claim 1, wherein the core has a thickness of approximately 0.5 inches to approximately 1.5 inches.

13. The seal apparatus according to claim 1, wherein the core a recovery rate less than approximately 1 second.

14. The seal apparatus according to claim 1, wherein the core has a density less than approximately 0.5 g/cm².

15. The seal apparatus according to claim 1, wherein the core has a thickness of approximately 0.5 inches to approximately 1.5 inches, a recovery rate less than approximately 1 second, and a density less than approximately 0.5 g/cm².
